(19) United States
(12) Patent Application Publication
(10) Pub. No.: US 2007/0009470 A1
(43) Pub. Date: Jan. 11, 2007

(54) Topical/on-the-skin estrogen cream, lotion, ointment, hair tonic, shampoo and wound healing powder

(76) Inventor: Kin Jung, Ventura, CA (US)

Correspondence Address: Kin S. Jung, MD, 695 Via Cielito, Ventura, CA, 93003-1236, US

(21) Appl. No.: 11/475,797

(22) Filed: Jun. 27, 2006

Related U.S. Application Data
(60) Provisional application No. 60/595,451, filed on Jul. 6, 2005.

(51) Int. Cl. A61K 8/64

(52) U.S. Cl. 424/70.14

(57) ABSTRACT

The Claimed Invention is the topical-to-skin use of generic estradiol, estriol, conjugated estrogens, or any other synthetic estrogen within a non-absorbable generic vehicle of cream, lotion, ointment, shampoo, hair tonic, and wound-healing powder, having no absorbents or deconstructors in their formulas. This invention causes skin basal cells to produce increased quantities of collagen and to retain skin-water resulting in increased skin-thickness. This increased thickness results in improved resilience, resistance to injury, trauma and infection. The target of this invention is the skin and skin only. Specifically, there is no intended absorption of hormone into the circulation to result in side effects or risk. Any possible unintended and minimal quantity of hormone absorbed would be metabolized normally by the liver and excreted in the urine without risk or awareness to the user. Improving skin health and healing existing injuries/disease can save individuals from mid 30's and older, and society in general millions of dollars annually in skin-related health care costs.

The Invention is Topical-To-Skin Delivered Estrogen (estradiol, estriol, conjugated estrogens and any synthetic estrogen) in very low dose quantities insufficient to penetrate the full thickness of the skin. This Invention results in no or minimally measurable sub-therapeutic systemic levels. The effect of the Topical-To-Skin Estrogen Invention is within the skin itself and is to not have an effect within the body as caused by significant and intended absorption of existing products and inventions.

The uniqueness of this Invention is its topical-to-the-skin, scalp and wounds application of estrogen delivered in vehicles of non-absorbable cream, lotion, ointment, shampoo, hair tonic and powder. These topical-on-the-skin Inventions are made from estradiol, estriol, conjugated estrogens or any other synthetic or natural estrogen and added to their generic bases readily made by any and all cosmetic and pharmaceutical companies including local pharmacies with existing technology and equipment and without the need to retool or redesign equipment. The percentages of the active ingredients vary from 0.001 to 10.0% and are readily formulated and produced by the manufacturer in the specification required for purpose specific.

The purpose of the Invention is (a) to thicken the skin of individuals from mid-30 years and older, and especially, the elderly with their thinned, parchment-like brittle, easily torn, injured and subsequently easily bruised or infected skin. The benefit to hospital, nursing home, sedentary individuals and others at risk of skin injury or infection is inestimable, saving the taxpayers millions of dollars annually. (b) The improved healing of wounds (surgical or accidental), skin ulcers, burns, abrasions, nicks, scrapes, etc., and their (c) improved resistance to such injuries and infections when used preventively, reduces time spent in healing and recuperation and reducing their associated costs, a true societal benefit.

### CROSS-REFERENCE TO RELATED APPLICATIONS

### Prior Art and Patents

Examples noted below are of already approved estrogen preparations for systemic (into the body) absorption using oral tablets and intravaginal tablets, creams, rings and transdermal (through the skin) delivery systems. As of this date, there are no approved or patented estrogen-containing products that purport our unique claim of topical-on-the-skin Invention for exclusive intradermal (within the skin) target effect and not to have an intended systemic target effect. All existing transdermal products (which pass through the skin) are intended for absorption into the body and to have a systemic/body effect, none of which meet the claims of this Invention on application. The existing products, absorbed into the body, have been safe for their intended use and have been FDA approved for many years.
1. Oral Estradiol “Activella” by Novo Nordisk Pharmaceuticals, Inc., 100 Collage Rd. West, Princeton, N.J. 08540. NDC 0169-5174-02.
2. Estradiol Transdermal System “Climara” by Berlex, Montville, N.J. 07045, made by 3M Pharmaceuticals, St. Paul, Minn. 55144. NDC 50419-454-04, et. al.
3. Estradiol Topical Emulsion “Estrasorb” by Novavax, Inc., Columbia, Md. 21046. NDC 66500-259-00, et. al.
4. Estradiol Intravaginal Ring “Estring” by Pharmacia Corp, Kalamazoo, Mich. 49001. NDC 0013-2150-36.
5. Estradiol Topical Gel “Estrogel 0.06%” by Unimed Pharmaceuticals, Inc., a Solvay Pharmaceuticals Inc. company, Marietta, Ga. 30062. NDC 0051-1028-58.
6. Estradiol Transdermal System “Menostar” by Berlex, Montville, N.J. 07045, made by 3M Pharmaceuticals, St. Paul, Minn. 55144. NDC 50419-455-04.
7. Estradiol Vaginal Tablets “Vagifem” by Novo Nordisk Pharmaceuticals, Inc., Princeton, N.J. 08540. NDC 0169-5173-03, et. al.
8. Estradiol Transdermal System “Vivelle” by Novartis Pharmaceuticals Corporation, East Hanover, N.J. 07936. NDC 0078-0348-42.
9. Estradiol Transdermal Patch “Alora ETS Patch” by Watson Laboratories, Inc., 311 Bonnie Circle, Corona, Calif. 92880. Generic Product.
10. Estradiol Vaginal Cream “Estrace Vaginal Cream” by Warner Chilcott, Inc., 100 Enterprise Drive, Rockaway, N.J. 07866. NDC 0072-0260-12.
11. Estradiol Oral Tablets “Gynodiol” by Novavax, Inc., Columbia, Md. 21046 NDC 66500-102-01, et. al.
12. Estradiol Acetate Oral Tablets “Femtrace” by Warner-Chilcott, Inc., 100 Enterprise Drive, Rockaway, N.J. 07866 NDC 0072-0260-14.
13. Estradiol Acetate Vaginal Ring “Femring” by Waner-Chilcott, Inc., 100 Enterprise Drive, Rockaway, N.J. NDC 66500-102-06, et. al.
14. Estrdiol Cypionate and MPA Monthly Injection (into muscle) “Lunelle Monthly Injection” by Pharmacia & Upjohn Company, Kalamazoo, Mich. 49001. NDC 0009-3484-04 et. al.
15. Conjugated Estrogens Vaginal Cream “Premarin Vaginal Cream” by Wyeth Pharmaceuticals, Inc., Philadelphia, Pa. 19101. NDC 0046-0872-01.
16. Ethinyl Estradiol/Norelgestromin Transdermal System “Ortho Evra” by Ortho-McNeil Pharmaceutical, Inc., Raratin, N. J. 08869 NDC 631-10-662-2.

### BACKGROUND OF THE INVENTION

Because of the long known benefit of estrogen to skin and hair health, the products were conceived following 30 years of clinical experience using them to improve the health and condition of many individuals' skin. The obvious was that younger individuals had healthier skin and hair than older ones. Women's skin and hair aged significantly faster as their hormone function diminished through menopause. Men's course of aging was usually slower due to less dependency upon their endogenous estrogen levels. Women's skin rejuvenated significantly as their hormone levels were normalized to pre-menopausal levels. Women's skin and hair aged significantly slower if they began oral hormone replacement therapy within 1 to 6 months following their last menstrual period.

It became evident and well documented in our clinical records that the skin of both men and women became thicker, more hydrated (moist), resistant to injury, sunburn and infection, diminished wrinkles, less bruising and healed faster when the estrogen containing cream or lotion was applied to those areas. Older individuals in particular demonstrated a significant improvement in their skin, resulting in fewer physician office visits for skin-related injuries or infections and superficial skin cancers. It is apparent that hormone replacement from within (by oral pills or by transdermal or vaginal delivery systems) is not as beneficial to the skin as hormone replacement from without (topical-to-skin cream, lotion, etc.) and thus the genesis of this application.

Due to the intended non-absorption into the body of these very minimal estrogen-containing products and by the very nature of their concentrations being sub-therapeutic (unable to provide a systemic response or effect) as proven in all studies domestic and foreign by the pharmaceutical companies producing products for systemic (into the body) absorption and by USA and foreign university studies, there is no discernable risk or side effect likely because they contain no deconstructors or absorbent agent(s). Again, the target is the skin and hair and not the body as a whole. Please see references attached (0020, pages 10-15).

### Risks and Side Effects

Any side effect most likely to happen would be the individual user's allergy, known or unknown, to any one of the components within the generic vehicles of cream, lotion, hair tonic, ointment, etc. Warnings to that effect would accompany any product and advice to consult medical attention immediately as deemed necessary by the user or his advisor. The minimal likelihood of an allergic response is the same as with all over-the-counter approved basic creams, ointments, lotions, shampoos, and powders. There are no known allergies to estriol, estradiol or conjugated estrogens (the active product ingredient or component) as these are existing compounds in everyone. Risks are nil.

### Metabolism

The sub-therapeutic systemic levels of estrogen delivered to the skin are of zero order kinetics and therefore are not of metabolic significance. Should any estrogen be absorbed against conventional knowledge by the average absorptive skin such sub-therapeutic quantities would be metabolized by the liver, as are all endogenous estrogens.

In both men and women (1:20 ratio of estrogen in men to women), circulating estrogens exist in a dynamic equilibrium of metabolic interconversions. These transformations take place primarily in the liver. Estradiol is converted reversibly to estrone, and both can be converted to estriol, which is the major urinary metabolite. Estrogens also undergo enterohepatic recirculation via sulfate and glucuronide conjugation in the liver, biliary secretion of conjugates into the intestine, and hydrolysis in the gut followed by absorption.

In the postmenopausal woman, a significant proportion of the circulating estrogens exist as sulfate conjugates, especially estrone sulfate, which serves as a circulating reservoir for the formation of more active estrogens. Although the clinical significance has yet to be determined, estradiol does not go through the first pass liver metabolism.

Estrogen within the skin is metabolized by oxidation and hydrolysis to inactive broken steroidal rings which eventually pass into the systemic circulation as individual components which are used by the liver for its own steroidal synthesis of numerous natural products, benefiting from the building blocks submitted. There appears to be no cumulative effect within the skin itself, of estrogen beyond 96 hours, even when intended by the significantly higher levels of estradiol, estriol or conjugated estrogens in the therapeutic transdermal (through-the-skin) delivery systems placed on the skin for 24 hours.

The rapid decline in the detectable levels of estrogen applied at therapeutic absorptive levels requires daily administration of those approved products listed above (0004: 2-13, 15-16), monthly injections into the muscle of a depository preparation of highly concentrated estrogen (0004: 14), or transdermal systems (patches) applied to skin daily or weekly, all proven safe.

With already submitted in existing approved patents estradiol metabolic curves established at varying doses of systemic absorbed levels sufficient to achieve hormone replacement levels or contraceptive pituitary gland suppressive levels, a steady state is achieved by about day 14 of continued use. These levels must be maintained by a continual source (oral, transdermal system, gel, intravaginal creams or rings, etc.) of estradiol at exponential doses from those proposed for the intradermal (within the skin) purpose intended in this application.

The intradermal levels of estradiol sufficient to achieve the desired effect are 0.001 to 0.015 mgm/5 ccs of vehicle, nearly homeopathic levels. If the products were used daily to achieve steady state by day 14, the measurable quantity of estradiol or estrone would be within the normal range of anticipated endogenously (within the body) naturally generated levels of estradiol and estrone, thus no measurable difference from that of individuals not topically treated with the Invention. Again, the target organ is the skin and hair only, thus no observable systemic (within the body) change in hormone levels.

The skin appears to benefit from topical-to-skin application much better than hormone replacement from within (by any approved method) and absent any of the systemic side effects, a true benefit of the Invention

### Mechanism of Action

It has long been known and understood that estrogen has a rejuvenating effect upon the skin and mucous membranes. Estradiol, estriol, and conjugated estrogens (especially topical-to-skin preparations) affect the lowest of the four levels of the skin (basal cells) by stimulating them to produce more collagen. This results in an attraction of water molecules and an increased hydration (fluid retention) and skin thickness. The improved and naturalization of the skin hydration results in an improved ability of the body's defensive white blood cells, fibroblasts and antibodies to migrate more freely within the skin's improved matrix. Molecular oxygen can now more easily diffuse through this well-hydrated environment and the skin can defend and heal itself more readily.

The estrogen-induced improved skin thickness is now more elastic, supple and resistant to external trauma, injury and infection. As the body's largest organ, any improved skin function results in diminished disease, injuries and costs associated with those conditions. Studies demonstrating this have been done in universities both domestic and foreign with their outcomes published and noted in the references attached.

It is the intent of this application to provide a product that acts uniquely only upon the skin and its four layers without measurable systemic effects and their potential risk of documented side effects attributed to systemically absorbed estriol, estradiol or conjugated estrogens. It is both the salutary effects upon the skin and the negligible risk of side effects that this product gives its user. The benefit of this Invention to society is inestimable as is the reduced cost to society in reduced skin injuries, infections, ulcers and superficial skin cancers.

### REFERENCES

### Bibliography and References

1. Estrogen and Hair Growth, Oh H S, Smart R C, Proc. Natl. Acad. Sci. USA Vol. 93, pp. 12525-12530, October 1996. Medical Sciences.
2. Skin Aging and Menopause: Implications for Treatment, Raine-Finning N J, Brincat M P, Muscat-Baron Y. American Journal of Clinical Dermatology: Vol. No. 6, 2003, pp. 371-378: Adis International.
3. Estrogen receptor beta is the predominant estrogen receptor in human scalp skin. Thornton M J, Taylor A H, Mulligan K, et. al, Experimental Dermatology, Vol. 12, No.2, April 2003, pp. 181-190.
4. Estrogens and the Hair Follicle, Conrad F, Paus R, German Soc. Dermatology, 2004 June 2: 412-23.
5. Treatment of Hair Loss, Price V H, Abdel-Salem M M, Greenspan J S, New England J Med. 1999, Vol. 341, No.13. P. 973.
6. Hair Cycle Control by Estrogens, Ohnemus U, Uenalan M, Conrad F, et. al. Endocrinology, 2005, Vol. 146, No. 3, pp. 1214-1225.
7. Controls of Hair Follicle Cycling, Stenn K S, Paus R, Physiological Reviews Vol. 81, No. 1, January 2001, pp. 449-494.
8. Large Induction of Keratinocyte Growth Factor Expression in the Dermis During Wound Healing (Topical Estrogen Stimulates KGF sic.), Proceedings of the National Academy of Sciences, 1992, Vol. 89, pp. 6896-6900.
9. Does estrogen prevent skin aging? (Yes, sic) (NHANES I), Dunn L B, Damesyn M, Moore A A, et al, Univ. Cal. At San Francisco, School of Medicine Archives of Dermatology, Vol. 133, No.3, March 1997.
10.Estrogen and Skin, An Overview, Shah, M G, Maibach H I, Am J Clin Dermatology, 2001; 2(3): 140-150.
11.Skin Collagen Changes in Postmenopausal Women Receiving Different Regimens of Estrogen Therapy, Brincat M, Versi E, Moniz C F, et. al, Obstetrics & Gynecology, 1987; 70:123-127.
12.Demonstration of estrogen receptors in the skin, Punnonen R, Lovgren T, Kouvonen I, Journal Endocrinological Investigation, 1980 Jul-Sept; 3 (3): 217-221.
13.Treatment of skin ageing . . . with estrogen compounds, Schmidt J B, Binder M, Macheiner W, Kainz C, Maturitas, 1994 Nov: 20(1): 25-30.
14. Topical Estrogen, Center for Clinical Age Management, Inc., by Topical Estrogen: Mar. 13, 2003, 10:00 pp.
15. Estrogen modulates cutaneous wound healing, J Clin Invest, 2003 May; 1. 111(9): pp. 1309-11.
16. Role for Estrogen In Wound Healing, Journal Watch, Vol. 1997, Issue 1118, 4 Nov. 18, 1997.
17. UF Researchers Report Estrogen Hasten Healing, Ross M F, Oct. 31, 1997 Gainesville, Fla.
18. Estrogen Accelerates Healing, Wounds, 2005: 17(11): 313-320
19. Hormonal Influences on Wound Healing: A Review of Current Experimental 2005; 17(11): 313-320.
20. Topical Estrogen and Wound Healing, The Analyst. Nov. 22, 2005
21. Topical Estrogen Accelerates Cutaneous Wound Healing in Aged Humans, Ashcroft G S, Geenwell-Wild T, Horan M A, et al., American Journal of Pathology: 1999, 155:1137-1146.
22. Hormonal Influences on Wound Healing: A Review of Current Experimental Data, Hardman M J, Ashcroft M A, Wounds 2005, 17(11) 313-320.
23. Potential Role of (Topical) Estrogens in Wound Healing, Ashcroft G S, Ashworth J J, American Journal of Clinical Dermatology, Vol. 4, No. 11, 2003: pp. 737-743.
24. Skin and Hormone Therapy, Clinical Obstetrics and Gynecology. 47 (2): 437-449: June 2004, Hall G, Phillips T J.
25. ©2005 Health Management Publications, Inc:
1. The Effects of Estrogen on Facial Skin Aging, Fuchs K O, Solis O, Tapawan R, Paranjpe J., Glens Falls Hospital, Glens Falls, N.Y. 12801. Cutis. 2003, June; 71(6): 481-8.
2. Hormonal Influences on Wound Healing: A Review of Current Experirnental Data, Wounds. 2005; 17(11): 313-320.
3. Martin P., Wound healing-aiming for perfect skin regeneration. Science. 1997; 276(5309): 75-81.
4. Ashcroft G S, Greenwell-Wild T, Horan M A, Wahl S M, Ferguson M W. Topical estrogen accelerates cutaneous wound healing in aged humans associated with an altered inflammatory response. Am J Pathol. 1999, 155(4):1137- 1146.
5. Ashcroft G S, Horan M A, Herrick S E, Tarnuzzer R W, Schultz G S, Ferguson M W.
6. Age-related differences in the temporal and spatial regulation of matrix metalloproteinases (MMPs) in normal skin and acute cutaneous wounds of healthy humans. Cell Tissue Res. 1997; 290(3): 581-591.
7. Thiboutot D, Jabara S, McAllister J M, Sivarajah A, Gilliland K, Cong Z, Clawson G. Human skin is a steroidogenic tissue: steroidogenic enzymes and cofactors are expressed in epidermis, normal sebocytes, and an immortalized sebocyte cell line (SEB-1). J. Invest Dermatol. 2003; 120(6): 905-914.
8. Margolis D J, Knauss J, Bilker W. Hormone replacement therapy and the prevention of pressure/decubitus ulcers and venous leg ulcers. Lancet. 2002, 359(9307): 675-677.
9. Herrick S, Ashcroft G, Ireland G, Horan M, McCollum C, Ferguson M. Up-regulation of elastase in acute wounds of healthy aged humans and chronic venous leg ulcers are associated with matrix degradation. Lab Invest. 1997, 77(3): 281-288.
10. Ashcroft G S, Yang X, Glick A B, et al. Mice lacking Smad3 show accelerated wound healing and an impaired local inflammatory response. National Cellular Biol. 1999; 1(5): 260-266.
11. Ashcroft G S, Lei K, Jin W, et al. Secretory leukocyte protease inhibitor mediates non-redundant functions necessary for normal wound healing. Nat Med. 2000; 6(10): 1147-1153.
12. Hardman M J, Ashcroft G S. New and alternative treatments for diabetic foot ulcers: hormones and growth factors. In: Boulton A J, Ed. The Foot in Diabetes. 4th ed. Chichester, UK: John Wiley & Sons (in press).
13. Brincat M P. Hormone replacement therapy and the skin. Maturitas. 2000, 35(2):107-117.
14. Ashcroft G S, Horan M A, Ferguson M W. Aging is associated with Reduced deposition of specific extracellular matrix components, an upregulation of angiogenesis, and an altered inflammatory response in a murine incisional wound healing model. Journal of Investigative Dermatology. 1997, 108(4): 430-437.
14. Ashcroft G S, Mills S J, Ashworth J J. Aging and Wound Healing. Biogerontology. 2002, 3(6): 337-345.
15. Ashcroft G S, Horan M A, Ferguson M W. Aging alters the inflammatory And endothelial cell adhesion molecule profiles during human cutaneous wound healing. Lab Invest. 1998, 78(1): 47-58.
16. Swift M E, Burns A L, Gray K L, DiPietro L A. Age-related alterations in the inflammatory response to dermal injury. Journal of Investigative Dermatology. 2001, 117(5): 1027-1035.
17. Ashcroft G S, Dodsworth J, van Boxtel E, et al. Estrogen accelerates Cutaneous wound healing associated with an increase in TGF-betal levels. Nat Med. 1997;3(11): 1209-1215.
18. Kovacs E J, Plackett T P, Witte P L. Estrogen replacement, aging, and cell mediated immunity after injury. J Leukoc Biol. 2004; 76(1): 36-41.
19. Ashcroft G S, Mills S J, Lei K, et al. Estrogen modulates cutaneous wound healing by downregulating macrophage migration inhibitory factor. J Clin Invest. 2003, 111(9): 1309-1318.
20. Kalaitzidis D, Gilmore T D. Transcription factor cross-talk: the estrogen receptor and NF-kappaB. Trends Endocrinol Metab. 2005; 16 (2): 46-52.
21. Kelly M J, Levin E R. Rapid actions of plasma membrane estrogen receptors. Trends Endocrinol Metab. 2001; 12 (4): 152-156.
22. Ashcroft G S, Mills S J. Androgen receptor-mediated inhibition of cutaneous wound healing. J Clin Invest. 2002; 110 (5): 615-624.
23. Im S, Lee E S, Kim W, et al. Expression of progesterone receptor in human keratinocytes. J Korean Med Sci. 2000; 15 (6): 647-654.
24. Ashworth J J, Smyth J V, Pendleton N, et al. The dinucleotide (CA) repeats polymorphism of estrogen receptor beta but not the dinucleotide (TA) repeat polymorphism of estrogen receptor alpha is associated with venous ulceration. Journal of Steroid Biochem Mol Biol. 2005; Sep 6 [Epub ahead of print].
25. Calandra T, Roger T. Macrophage migration inhibitory factor: a regulator of innate immunity. National Rev Immunol. 2003; 3 (10): 791-800.
26. Yu C M, Lai K W, Chen Y X, Huang X R, Lan H Y. Expression of Macrophage migration inhibitory factor in acute ischemic myocardial injury. J Histochem Cytochem. 2003, 51(5): 625-631.
27. Hardman M J, Whaite A, Zeef L, Burow M, Nakayama T, Ashcroft G S. Macrophage migration inhibitory factor: a central regulator of wound healing. Am J Pathol. In press.
28. Gilliver S C, Wu F, Ashcroft G S. Regulatory roles of androgens in cutaneous wound healing. Thromb Haemost. 2003, 90 (6); 978-985.
29. Ashcroft G S, Mills S J, Flanders K C, et al. Role of Smad3 in the hormonal modulation of in vivo wound healing responses. Wound Repair Regen. 2003, 11(6): 468-473.
30. Taylor R J, Taylor A D, Smyth J V. Using an artificial neural network to predict healing times and risk factors for venous leg ulcers. Journal of Wound Care. 2002; 11 (3): 101-105.
31. Orentreich N, Brind J L, Rizer R L, Vogelman J H. Age changes and sex differences in serum dehydroepiandrosterone sulfate concentrations throughout adulthood. J Clin Endocrinol Metab. 1984, 59(3): 551-555.
32. Mills S J, Ashworth J J, Hardman M J, Ashcroft G S. The sex steroid precursor DHEA accelerates cutaneous wound healing via the estrogen receptor. Journal of Investigative Dermatol. In press.
33. Rossouw J E, Anderson G L, Prentice R L, et al; Writing Group for the Women's Health Initiative Investigators. Risks and benefits of estrogen plus progestin in healthy postmenopausal women: principal results From The Women's Health Initiative randomized controlled trial. JAMA. 2002; 288(3): 321-333

### BRIEF SUMMARY OF THE INVENTION

The Invention is estradiol, estriol, conjugated estrogens or other synthetic estrogens in a non-absorbable generic vehicle of cream, lotion, ointment, hair-tonic, shampoo and powder that is applied-to-the-skin (topical) and targets the skin and the skin only. There is no intended pass-through-the-skin for absorption into the systemic circulation as is the intent of all existing inventions and technology. The Invention resides in and is effective within the skin only.

The Invention thickens the skin from within the skin itself by enhancing its hydration/fluid retention. This results from the stimulation of the lowest of the four levels of skin, the basal cells, to produce more collagen that acts as a sponge, attracting and holding water within the skin proper.

The Invention results in improved skin physiology and function by allowing the body's natural defenses of white blood cells, antibodies, and molecular oxygen to more readily pass through and within this improved highway matrix of collagen and water-within-the-skin proper. The “normalization” or “near-normalization” or any improvement whatsoever in the physiology of the skin results in diminished incidences of infections and injuries that accompany the natural skin-aging process.

The Invention thickens skin with a secondary benefit of diminished wrinkles and an improved cosmesis to wherever the Invention is applied (hands, arms, face, neck, buttocks, thighs, etc.). The improved skin thickness results in less frequent injuries and infections that cannot be overemphasized. This results from the natural improvement in skin physiology by the use of this Invention.

Using existing technology, the Invention can be produced by all cream, lotion, ointment, powder, hair-tonic manufacturers and pharmaceutical companies. There is no need to retool or to invest in additional equipment thereby costing minimal to enter the market, using this Invention. Existing inventions make none of the above claims.

### DETAILED DESCRIPTION OF THE INVENTION

The invention is estradiol, estriol, conjugated estrogens and other synthetic and semi-synthetic estrogens in a non-absorbable generic vehicle of cream, lotion, ointment, shampoo, hair-tonic or powder. The product is intended for topical-to-skin use only and its effect is within the skin only and is not intended for systemic effect or for absorption within the body. To this date, all approved estradiol, estriol or conjugated estrogens are for systemic absorption resulting in measurable therapeutic levels, which this invention does not intend nor give.

Estradiol is a white crystalline powder, chemically described as estra-1,3,5 (10)-triene-3-17β-diol. It has an empirical formula of C₁₈H₂₄O₂ and a molecular weight of 272.39. Estradiol metabolizes naturally and physiologically to estriol and estrone. 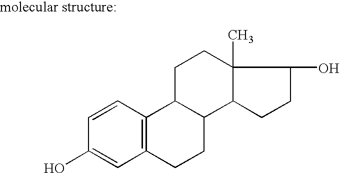
molecular weight of estradiol is 272.39.

1. The PROBLEM: The natural aging of the skin, the body's largest organ, becomes apparent from the mid-30's to early 40's and beyond. The normal skin aging physiology evidences as skin gradually becoming thinner, dryer, more wrinkled, more easily bruised, injured and infected, and susceptible to superficial skin cancers. Very notable is the tendency of the skin and the tissues under the skin to become more easily infected as evidenced by swelling, pain, redness and increased heat of the infected skin and adjacent areas. Decreased function and mobility occur as a result of these changes. Work or recreation time is lost at significant societal cost. As hours to days progress delaying medical treatment of the skin injury, the difficulty in treating efficiently and effectively diminishes as the risk of dissemination of the skin infection into the body becomes real and dangerous with hospitalization becoming likely.
These once thought “simple skin injuries” today and in the very near future are becoming more difficult to treat and to obtain an excellent or desirable outcome. There are two major reasons for this dilemma. (1) The effectiveness of antibiotics is becoming less as drug resistance increases and the body's natural first defense, (2) the skin, becomes weaker with age. This happens because the skin becomes thinner, more brittle, and more easily bruised, injured torn and infected. These are all natural processes that can be prevented or reversed in great part when treated preventively and/or therapeutically with this Invention.

2. The SOLUTION: (claim 1) This Invention thickens the skin from within itself by normalizing its natural moisture/hydration, increasing its collagen content, and (claim 2) improving the skin matrix through which the body's white blood cells, antibodies and fibroblast cells can (claim 3) normalize or improve its anti-infective and strengthening defenses. This “normalization” or “near-normalization” of the skin results in (claim 4) diminished injuries and infections. (claim 5) Skin healing is greatly enhanced and (claim 6) time-to-recovery greatly reduced, allowing return-to-work or other normal activity sooner than later. (claim 7) Lost time from work-related skin injuries and infections can be reduced, (claim 8) saving millions of dollars in workers' compensation claims and payouts.
These injuries and infections account for millions of dollars in annual health care and insurance costs including Medicare and Medi- (any State-specific) charges. These costs will accelerate exponentially in the near future due to the graying of America, the increasing overall population, and the diminishing efficacy of the later generations of antibiotics. The Invention (claim 9) reduces the risk of skin injuries and infections and their attendant costs to society in time-off-work and hospitalizations for severe skin infections and injuries.
Look at any maturing individual and see how the skin becomes easily bruised and injured and eventually becomes a very thinned and damaged body-envelope against the external environment and a poor container of the internal environment. The later results in the gradual seepage of fluids (serum and whole blood) from any crack in this armor to the development of within-the-skin and below-the-skin infections, usually requiring medical attention and costs.
Since years 2000-2001, the risk of bacterial infections becoming resistant to antibiotics has become a reality. This risk translates to significant morbidity (of illness and disease causing dysfunction and pain) and mortality to the aging populations and demographics of our nation in particular and to the global community in general. This Invention addresses both preemptive and curative measures, (claim 10) mitigating those costs in great part these skin related challenges and saving society millions of dollars annually.
